(19) United States
(12) Patent Application Publication
(10) Pub. No.: US 2019/0054008 A1
(43) Pub. Date: Feb. 21, 2019

(54) COMPOSITIONS AND METHODS FOR EXTRACTION OF BOTANICAL COMPOUNDS FROM PLANTS

(71) Applicant: AMYRIS, INC., Emeryville, CA (US)

(72) Inventor: Diva CHAN, Emeryville, CA (US)

(73) Assignee: AMYRIS, INC., Emeryville, CA (US)

(21) Appl. No.: 15/763,029

(22) PCT Filed: Sep. 22, 2016
(86) PCT No.: PCT/US16/53111

§ 371 Date: Mar. 23, 2018

Related U.S. Application Data
(60) Provisional application No. 62/222,467, filed on Sep. 23, 2015.

(51) Int. Cl. A61K 8/9789; A61K 41/00; A61K 36/738; A61K 36/53; A61Q 19/00; A61Q 5/00

(52) U.S. Cl. CPC A61K 8/9789; A61K 41/0004; A61K 36/738; A61K 36/53; A61K 2236/35; A61Q 5/00; A61K 2236/15; A61K 2236/17; A61K 2800/805; A61Q 19/00

(57) ABSTRACT

The present invention provides methods, compositions and kits for extracting botanical compounds from plant parts using a carrier medium comprising a bio-based farnesene, a hydrocarbon composition derived from the bio-based farnesene or a combination thereof. In certain embodiments, rose petals are used as a plant material, and a squalane composition derived from bio-based farnesene is used as a carrier medium.

### 1. CROSS REFERENCE TO RELATED APPLICATION

This application claims benefit of priority to U.S. Provisional Patent Application No. 62/222,467, filed Sep. 23, 2015, which is incorporated herein by reference.

### 2. FIELD

The methods and compositions provided herein generally relate to extracting botanical compounds, in particular volatile odorous compounds, from plants or plant parts using a carrier medium comprising a bio-based farnesene, a hydrocarbon composition derived from the bio-based farnesene, or a combination thereof.

### 3. BACKGROUND

Plants are rich sources of volatile odorous compounds which impart characteristic fragrances. The volatile odorous compounds and other botanical compounds can be extracted using a variety of extraction methods. One of these methods includes infusion extraction, wherein a carrier oil is added to plant parts to draw out botanical compounds into the carrier oil. For example, flowers, leaves, roots, and other plant parts can be added to a carrier oil, and the mixture can be heated to transfer botanical compounds including volatile odorous compounds in these plant parts into the carrier oil to produce an end product with a desired fragrance profile or other beneficial properties.

While the process of making an infused oil product can be relatively simple, it is difficult to extract a high concentration of botanical compounds from plant parts into the carrier oil. Plant cells are surrounded by thick, rigid cell walls in addition to a cell membrane inside the wall. The cell walls are also tightly bonded together to form the structure of a plant. Therefore, depending on the plant source, it is not easy to break down the plant parts without a harsh chemical or mechanical treatment to release botanical compounds from the cells. In addition, many botanical compounds, such as volatile odorous compounds, exist in small quantities in plants. As such, it is difficult to produce infused oil products that retain the fragrance profiles of natural sources as they exist in nature. It is also difficult to fully extract available botanical compounds with beneficial properties into carrier oils. Therefore, there is a need to improve methods for efficiently extracting botanical compounds from plant parts and for producing infused end products that preserve the fragrance profiles and purity of botanical compounds as they exist in nature.

Embodiments of the present invention meet these and other needs.

### 4. SUMMARY

Provided herein are compositions and methods for producing a high quality botanical infused product comprising botanical compounds from plant materials. In the methods provided herein, botanical compounds which naturally exist in plant materials are extracted into a carrier medium. Without being bound by theory, it is believed that when a bio-based farnesene or a hydrocarbon composition derived from the bio-based farnesene is used as a carrier medium, the carrier medium can impregnate the matrix of plant materials and extract botanical compounds at a higher quality and/or quantity, compared to other comparable carrier media. As used herein, a bio-based farnesene refers to farnesene which is produced by fermentation by converting renewable carbon sources, such as sugar, by microorganisms. In particular, when a squalane composition derived from the bio-based farnesene is used as a carrier medium, it is capable of extracting botanical compounds from plant materials at a higher quality and/or quantity compared to squalanes obtained from other sources such as olives. For example, the intensity of fragrance from botanical compounds obtained with the present methods is generally higher than that obtained with other industry accepted carrier oils. Thus, the present extraction methods and compositions can be used to produce a higher quality botanical infused end product capable of emitting fragrance that closely resembles that found in its natural plant source.

In one aspect, provided herein is a method of extracting botanical compounds from a plant material comprising: (a) contacting a plant material comprising one or more botanical compounds with a carrier medium to produce a mixture, wherein the carrier medium comprises a bio-based farnesene, a hydrocarbon composition derived from the bio-based farnesene, or a combination thereof; and (b) incubating the mixture to extract the one or more botanical compounds from the plant material into the carrier medium to produce a botanical infused product.

In certain embodiments, the hydrocarbon composition of the carrier medium comprises a C15 hydrocarbon, a C30 hydrocarbon, or a combination thereof. In certain embodiments, the hydrocarbon composition in the carrier medium is bio-based farnesene. In certain embodiments, the hydrocarbon composition in the carrier medium comprises farnesene derived from bio-based farnesene. In certain embodiments, the hydrocarbon composition in the carrier medium comprises partially hydrogenated farnesene derived from bio-based farnesene. In certain embodiments, the hydrocarbon composition in the carrier medium comprises a squalane composition derived from bio-based farnesene. In certain embodiments, the hydrocarbon composition in the carrier medium comprises squalane and isosqualane derived from bio-based farnesene. In certain embodiments, the hydrocarbon composition in the carrier medium comprises squalane, isosqualane and neosqualane. In certain embodiments, the hydrocarbon composition in the carrier medium comprises a farnesene dimer. In certain embodiments, the hydrocarbon composition in the carrier medium comprises a farnesene dimer. In certain embodiments, the hydrocarbon composition in the carrier medium comprises any combination of C15 or C30 hydrocarbons described herein.

In another aspect, provided herein are botanical infused products comprising one or more botanical compounds infused into a carrier medium comprising a bio-based farnesene or a C15 or C30 hydrocarbon composition produced from the bio-based farnesene. In certain embodiments, a kit is also provided. The kit may comprise a container comprising a botanical infused product provided herein and instructions for using the botanical infused product. The kit may further comprise a wipe that is suitable for impregnation with the botanical infused product. The kit may further comprise an additional container comprising a diluent which may be used to dilute the botanical infused product.

### 5. BRIEF DESCRIPTION OF THE FIGURE

FIG. 1 illustrates results of the ranking test of three extraction samples described in Example 2: a squalane composition derived from bio-based farnesene (also referred to as bio-based squalane) as a carrier medium to extract botanical compounds from rose petals; olive derived squalane as a carrier medium to extract botanical compounds from rose petals; and jojoba oil as a carrier medium to extract botanical compounds from rose petals. The results show the assessors' selection of sample rank by floral intensity from strongest (1) to weakest (3).

### 6. DETAILED DESCRIPTION OF THE EMBODIMENTS

### 6.1 Definitions

When referring to the compounds, compositions and methods provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

As used herein, the term “plant material” refers any parts or tissues of a plant, or a whole plant, including but not limited to, flowers, leaves, stems, fruits, seeds, roots, stalks, barks, carpels, stamen, petals, and the like.

As used herein, the term “botanical compound” includes any compound that can be extracted from plants or plant parts. The botanical compounds, as used herein, can include compounds that provide beneficial properties to the skin, hair or nails. The botanical compounds can also include volatile compounds, such as volatile odorous compounds, which are responsible for the particular fragrance profile for the plant parts.

As used herein, the term “volatile odorous compound” refers to any organic compound capable of evaporating at room temperature and which is responsible for the odor or scent.

As used herein, the term “carrier medium” is a substance which is mixed with a plant material to draw out botanical compounds from the plant material. A carrier medium is typically a liquid at room temperature but can also be in solid, semi-solid or gas state.

As used herein, the term “bio-based farnesene” refer to farnesene which is biologically produced from microorganisms, in particular, genetically modified microorganisms, during fermentation of renewable carbon sources such as sugar.

As used herein, the term “a hydrocarbon composition derived from bio-based farnesene” refers to a composition comprising an organic compound comprised of carbon and hydrogen which is produced from the bio-based farnesene by catalytic reaction, thermal reaction, hydrogenation, or any combination thereof.

As used herein, “infused product” refers to an end product comprising a carrier medium mixed together with botanical compounds from a plant material.

“Farnesene” as used herein refers to α-farnesene, β-farnesene or a mixture thereof. “α-farnesene” refers to a compound having the following structure:

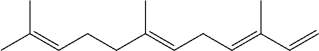

or a stereoisomer thereof.

“β-farnesene” refers to a compound having the following structure:

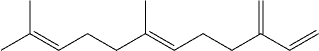

or a stereoisomer thereof. In some variations, β-farnesene comprises a substantially pure stereoisomer of β-farnesene. In other variations, β-farnesene comprises a mixture of stereoisomers, such as cis-trans isomers. In further embodiments, the amount of each of the stereoisomers in the β-farnesene mixture is independently from about 0.1 wt. % to about 99.9 wt. %, from about 0.5 wt. % to about 99.5 wt. %, from about 1 wt. % to about 99 wt. %, from about 5 wt. % to about 95 wt. %, from about 10 wt. % to about 90 wt. %, from about 20 wt. % to about 80 wt. %, based on the total weight of the β-farnesene mixture.

“Farnesene” refers to a compound having the following structure:

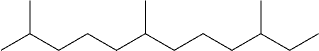

or a stereoisomer thereof.

“Hydrogenated farnesene” refers to farnesene (e.g., β-farnesene) wherein at least one carbon-carbon double bond is hydrogenated. Hydrogenated farnesene encompasses, for example, β-farnesene in which one, two, three or four double bonds are hydrogenated. Hydrogenated farnesene is obtained by complete or partial hydrogenation of farnesene, and encompasses farnesene.

“Partially hydrogenated farnesene” refers to farnesene (e.g., β-farnesene) wherein one, two, or three double bonds are hydrogenated. Partially hydrogenated farnesene can be obtained by partial hydrogenation of farnesene. In some embodiments, a composition comprising partially hydrogenated farnesene (e.g., obtained by partial hydrogenation of farnesene) may include amounts of farnesene and/or farnesene in addition to one or more of dihydrofarnesene, tetrahydrofarnesene and hexahydrofarnesene.

As used herein, the term “dihydrofarnesene” refers to farnesene in which one double bond is hydrogenated.

As used herein, the term “tetrahydrofarensene” refers to farnesene in which two double bonds are hydrogenated.

As used herein, the term “hexahydrofarnesene” refers to farnesene in which three double bonds are hydrogenated.

As used herein, “squalane” refers to a compound having the following formula:

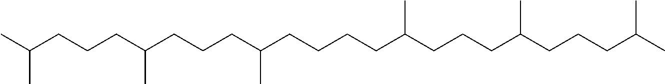 [SMILES: CC(C)CCCC(C)CCCC(C)CCCCC(C)CCCC(C)CCCC(C)C]

or a stereoisomer thereof.

As used herein, “iso-squalane” or “isosqualane” refers to a compound having the following formula:

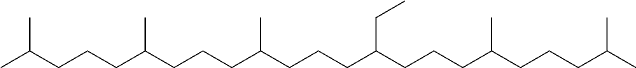 [SMILES: CCC(CCCC(C)CCCC(C)C)CCCC(C)CCCC(C)CCCC(C)C]

or a stereoisomer thereof.

As used herein, “neosqualane” refers to a compound having the following formula:

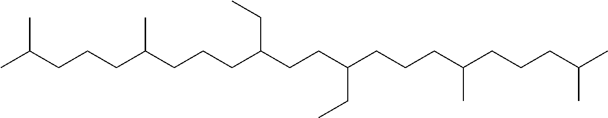 [SMILES: CCC(CCCC(C)CCCC(C)C)CCC(CC)CCCC(C)CCCC(C)C]

or a stereoisomer thereof.

As used herein, the term “farnesene dimer” refers to compounds having the following formula:

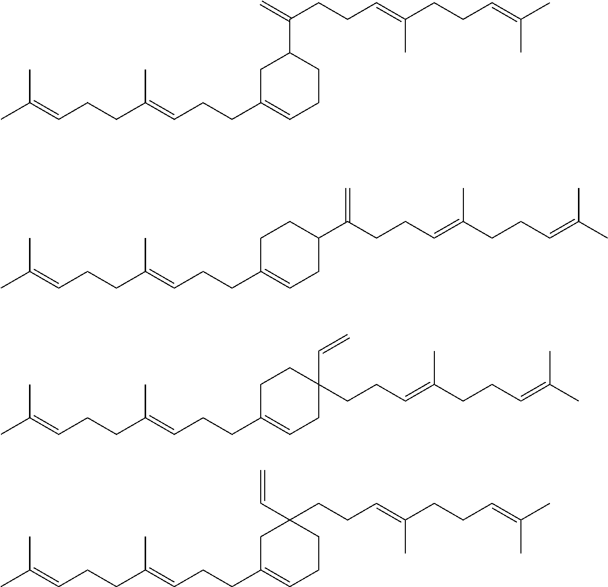

or stereoisomers thereof.

As used herein, the term “farnesene dimer” refers to compounds having the following formula:

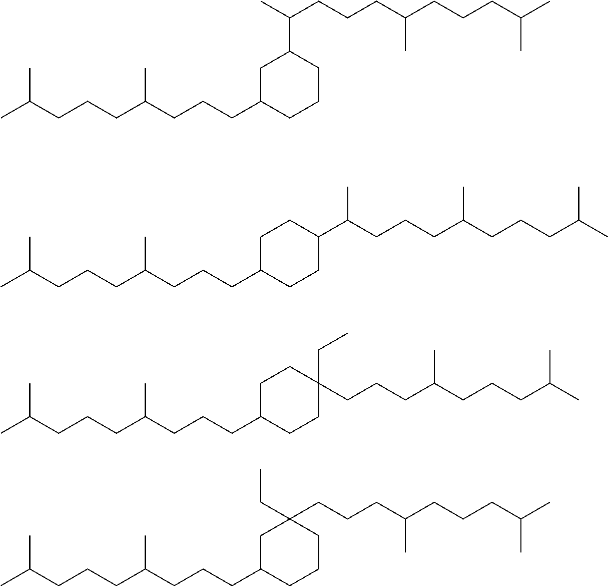

or stereoisomers thereof.

As used herein, % with reference to hydrocarbon compositions refers to % measured as wt. % or as area % by GC-MS or GC-FID, unless specifically indicated otherwise.

The term “substantially free of” or “substantially in the absence of,” when used in connection with an article (including, but not limited to, a compound or composition comprising a compound), refers to the article that includes at least about 85% or about 90% by weight, in certain embodiments, about 95%, about 98%, about 99%, or about 100% by weight, of the designated article.

In the following description, all numbers disclosed herein are approximate values, regardless of whether the word “about” or “approximate” is used in connection therewith. Numbers may vary by 1%, 2%, 5%, or by 10 to 20%. Whenever a numerical range with a lower limit R^{L} and an upper limit R^{U} is disclosed, any number falling within the range is specifically disclosed. In particular, the following numbers Rₖ within the range are specifically disclosed: Rₖ=R^{L}+k*(R^{U}—R^{L}), wherein k is a variable ranging from 0.01 to 1 with a 0.01 increment, i.e., k is 0.01, 0.02, 0.03, 0.04, 0.05, . . . , 0.5, 0.51, 0.52, . . . , 0.95, 0.96, 0.97, 0.98, 0.99, or 1. Further, any numerical range defined by any two numbers Rₖ as defined above is also specifically disclosed herein.

The term “a,” “an,” and “the” means “at least one” unless the context clearly indicates otherwise.

### 6.2 Methods for Extraction

In one aspect, provided herein is a method of extracting botanical compounds from a plant material into a carrier medium. In some embodiments, the carrier medium comprises a bio-based farnesene, a C15 or C30 hydrocarbon composition derived from the bio-based farnesene, or any combination thereof. The bio-based farnesene and hydrocarbons derived therefrom are further described in detail in Section 6.3 below.

Examples of plant materials suitable for extraction include flowers, leaves, stems, fruits, seeds, roots, stalks, barks and the like. The plant materials can be derived from any natural plant sources which are known to emit scent. These include, but are not limited to, rose petals, jasmine flowers, orange blossom, grapefruit blossom, lime blossoms, nectarine blossom, calendula, calendula flowers, peony, lily, bluebell, lavender, gardenia, marigold, daffodil, verbena, linden, chamomile, geranium, arnica, calendula, basil, sage, ginger, ginseng, cacao, nutmeg, anise, sage, and vanilla beans. Any suitable plant material, either a whole plant or plant part, can be used for extraction.

In certain embodiments, the source of plant materials may be selected based on their medicinal or homeopathic properties. As an example, chamomile and lavender flower heads may be infused with a carrier medium provided herein, and the botanical infused product can be used as a calming and soothing oil. In another example, rose petals are also known for containing compounds that soothe and soften skin. In yet another example, arnica flowers may be selected to produce a botanical infused product for relieving pain from bruises. Calendula flowers may be used to produce botanical infused products for cuts, scrapes, and insect bites. Ginseng may be infused with a carrier medium to produce a botanical infused product that provides anti-inflammatory properties to the skin. Other plant materials suitable for treating various skin conditions, such as acne, eczema, psoriasis, and the like, are further described in Pharmacogn Rev. 8(15): 52-60 (2014).

One of skill in the art will appreciate that the plant materials should be harvested with care and in a way to preserve fragrance profiles of botanical compounds. When the plant parts, such as flowers and leaves, are picked, they can be very fragile. In some embodiments, the plant parts are processed within about 24 hours, typically within about 12 hours, more typically within about 6 hours, even more typically within about 3 hours to prevent degradation and to preserve the quality of botanical compounds, in particular volatile odorous compounds, in the plant parts.

In certain embodiments, freshly picked plant parts can be directly added to a carrier medium for infusion extraction. In other embodiments, the plant materials can be processed prior to contacting them with a carrier medium. For example, the plant material can be de-stemmed, peeled or de-seeded. In another example, the plant material can be pulverized (e.g., masticated, chopped, minced, grounded, or scored) to release cellular contents into the carrier medium. In some embodiments, the plant material can be pulverized after harvest. In some embodiments, the plant material can be partially or completely dried prior to mixing it into a carrier medium. In other embodiments, the whole plant can be added to the carrier medium.

In some embodiments, the method of extraction can also include contacting the prepared plant material with a carrier medium comprising a bio-based farnesene, a hydrocarbon composition derived from the bio-based farnesene, or any combination thereof. A bio-based farnesene refers to farnesene which is produced by fermentation of renewable carbon sources, such as sugar, using microorganisms which may be genetically modified to convert the renewable carbon sources into farnesene. Alternatively or additionally, the carrier medium can comprise a C15 or C30 hydrocarbon composition derived from the bio-based farnesene. These hydrocarbon compositions are produced using the bio-based farnesene as substrates and converting them to other C15 or C30 hydrocarbons by one or more combinations of processes, such as thermal, catalytic, and hydrogenation processes. Examples of C15 hydrocarbons derived from the bio-based farnesene include farnesene and partially hydrogenated farnesene, such as dihydrofarnesene, tetrahydrofarnesene, and hexahydrofarnesene. Examples of C30 hydrocarbons derived from the bio-based farnesene include squalane, farnesene dimers, and farnesene dimers.

Generally, the bio-based farnesene and/or hydrocarbon compositions derived therefrom comprise relatively pure C15 and/or C30 hydrocarbon content. In certain embodiments, the C30 hydrocarbon content in a composition derived from the bio-based farnesene comprises at least about 85%, at least about 90% or at least about 95% by weight of C30 hydrocarbons, based on the total amount of the hydrocarbon composition. Impurities (e.g., compounds other than C15 or C30 hydrocarbons) in the hydrocarbon composition are less than about 10% by weight, typically less than about 5% by weight, or typically less than about 2% by weight based on the total amount of the hydrocarbon composition. Without wishing to be bound by any theory, the purity of presently provided hydrocarbon compositions derived from the bio-based farnesene and their branching molecular structure may provide a superior matrix to impregnate plant materials and extract and stabilize botanical compounds. Furthermore, these hydrocarbon compositions contain extremely low levels of impurities and odor, and therefore, provide ideal carrier media which do not impart any odor of their own to the final products.

In certain embodiments, the carrier medium is entirely comprised of bio-based, i.e., consists essentially of, farnesene or C15 or C30 hydrocarbon compositions derived from the bio-based farnesene. In particular embodiments, the major proportion of the carrier medium comprises bio-based farnesene, C15 hydrocarbon compositions derived therefrom, and/or C30 hydrocarbon compositions derived therefrom. For example, the carrier medium comprises at least about 50%, at least about 60%, at least about 70%, at least about 80%, at least about 85%, at least about 90%, at least about 95%, at least about 96%, at least about 97%, at least about 98%, or at least about 99% by weight of bio-based farnesene, C15, and/or C30 hydrocarbon compositions derived from the bio-based farnesene. In other embodiments, however, the carrier medium may further comprise other components in addition to the C15 or C30 hydrocarbon composition. For example, solvents such as alcohol, water, emulsifier, or other substances which can assist in extracting botanical compounds from plant materials can be incorporated into the carrier medium. In certain embodiments, a carrier medium provided herein comprises less than about 50% by weight, less than about 20% by weight, less than about 10% by weight, or less than about 5% by weight of additional components other than the C15 or C30 hydrocarbon compositions.

One of ordinary skill in the art will recognize that any suitable amount of plant materials can be added to a carrier medium. In some embodiments, the proportion of plant materials and a carrier medium may depend on the intensity or concentration of scent desired in the infused end product. For example, if a higher concentration of volatile odorous compounds or intensity of scent is desired in the infused end product, then a smaller amount of carrier medium may be used per given amount of plant materials. In some embodiments, if it is desired to use the infused end product directly as a personal consumer product, then a larger amount of carrier medium may be used per given amount of plant materials. For example, the weight ratios between a plant material and a carrier medium may vary between 1:100 to 100:1 or 1:10 to 10:1. For example, about 10 grams to about 1000 grams, or about 50 grams to about 500 grams of dried or fresh plant materials may be added per 500 grams of carrier medium. In another example, the weight (e.g., gram) to volume (e.g., mL) ratio between a plant material and a carrier medium may vary between 1:100 to 100:1 or 1:10 to 10:1. These examples are merely illustrative, and any variations of suitable proportions will be readily apparent to those skilled in the art.

The present extraction method further includes incubating the mixture of a carrier medium and a plant material comprising botanical compounds. In certain embodiments, the mixture may be incubated under heat and/or pressure to increase the diffusion rate of the botanical compounds from the plant material into the carrier medium. For example, the mixture can be gently heated in a double boiler. In another example, the mixture can be placed in an oven at a relatively low temperature, generally less than about 200° C., less than about 100° C., less than about 75° C., less than about 50° C., or less than about 30° C. In yet another example, the mixture can be incubated between about 30° C. to about 50° C., or at about 40° C. In yet another example, the mixture in a closed container may be placed under solar radiation. Typically, the heat method is used for tough herbs, bark, roots and seeds. The duration of incubation may vary depending on the nature of plant materials and botanical compounds in the plant materials. For example, the duration of incubation may vary from a few hours to several days. For example, the duration of incubation may include about 1 to about 100 hours, about 12 to about 48 hours, or about 12 to about 24 hours. The incubation duration can affect the fragrance profile of the infused end product.

In certain embodiments, the cold method is used for infusion extraction. The cold method is a slow process typically used for delicate flower petals. For example, the mixture may be simply incubated in a container at room temperature, generally between about 15° C. to about 30° C., or even at a lower temperature between about 4° C. to about 15° C. In certain embodiments, the mixture can be incubated under vacuum without any air to prevent oxidation of botanical compounds. The duration of incubation may vary, typically ranging from a few hours to several weeks or even months. In certain embodiments, the mixture can be stirred or agitated periodically to increase the diffusion rate of botanical compounds from the plant material into the carrier medium. In certain embodiments, the mixture can be processed using enfleurage extraction methods (e.g., pressing between layers of glasses or other weights).

After incubating the plant material in a carrier medium for a suitable time period, in certain embodiments, the plant material may be removed from the mixture. For example, the plant material may be strained or filtered from the botanical infused products. In some embodiments, a small percentage of residual plant matter may remain in the botanical infused product after filtration. For example, about 0.1% to about 5%, or about 1% to about 3% by weight of residual plant matters may remain in the infused end product. In other embodiments, the plant material may not be filtered and remain together with the infused end product for aesthetics.

In certain embodiments, infusion extraction devices may be used to efficiently extract botanical compounds from plant materials. Examples of infusion extraction devices are described in, for example, U.S. Pat. Nos. 4,721,035 and 4,832,951, which are incorporated herein by reference in their entirety. Briefly, the devices described in these patents include two chambers and a piston. A piston operating in a first chamber draws a solvent into the first chamber where it may be heated. The heated solvent is then moved into a second chamber which contains the infusible material, and where infusion extraction takes place. The piston then moves the solvent containing the extract through a filter into the first chamber. The extraction residues are left behind in the second chamber.

In addition to infusion extraction processes, other suitable methods may be used to extract botanical compounds from plant materials. For example, botanical compounds in plant materials can be extracted using steam distillation, super critical water treatment, and super critical CO₂ extraction methods known in the art. Some of these methods are described in, for example, U.S. Pat. No. 6,331,320 and U.S. Patent Application Publication No. 2013/0338241, which are incorporated herein by reference in their entirety. The extracts of botanical compounds obtained from other extraction methods can be mixed together with a carrier medium comprising bio-based farnesene or other hydrocarbon compositions derived therefrom. Mixing extracts or essential oils into the presently provided carrier medium can provide additional benefits such as a longer shelf life and persistency of odor compared to other carrier oils.

### 6.3 Bio-Based Farnesene and Hydrocarbon Compositions Derived from Bio-Based Farnesene

In another aspect of the invention, provided herein are bio-based farnesene and hydrocarbon compositions derived therefrom which can be used as a carrier medium for extraction of botanical compounds from plant materials. The bio-based farnesene used in embodiments of the present invention are produced from microorganisms, including bioengineered microorganisms, using a renewable carbon source, such as sugar. In particular embodiments, bio-based farnesene can be derived from a renewable carbon source using genetically modified microbial cells as described in U.S. Pat. No. 7,659,097 B2, U.S. Pat. No. 7,399,323 B2, U.S. Pat. No. 7,846,222 B2, U.S. Pat. No. 8,257,957 B2 or International Patent Publication WO2007/139924 A2, each of which is incorporated herein by reference in its entirety. The bio-based farnesene produced from fermentation of renewable carbons can be used as substrates to generate additional hydrocarbon compositions which are suitable as a carrier medium in the present extraction methods. The bio-based farnesene and other hydrocarbon compositions are particularly useful as carrier media as they are free or substantially free of impurities. For example, hydrocarbon compositions provided herein are free or substantially free of small, volatile, organic oxygenate compounds (e.g., alcohols, acids, aldehydes, 6-methyl-5-penten-2-one, or the like) which can cause an odor in the hydrocarbon compositions. These carrier media also exhibit low or no odor so as not to impact the overall odor profile of the final infused product.

### 6.3.1. C15 Hydrocarbon Compositions Derived from Bio-Based Farnesene

The bio-based farnesene derived from fermentation of renewable carbons can be used to generate additional C15 hydrocarbon compositions which are also suitable as a carrier medium for the present extraction methods. For example, the bio-based farnesene can be hydrogenated to produce farnesene or partially hydrogenated farnesene. The farnesene is the fully hydrogenated C15 compound of farnesene. The partially hydrogenated C15 compounds include dihydrofarnesene, where one double bond of farnesene is hydrogenated. During partial hydrogenation process, other partially hydrogenated farnesenes, such as tetrahydrofarnesene and hexahydrofarnesene, may be co-produced with dihydrofarnesene. Any one or combinations of C15 hydrocarbon compositions derived from bio-based farnesene may be used as a carrier medium to extract botanical compounds from the plant materials.

Generally, the production of other C15 hydrocarbon compositions using bio-based farnesene as substrates comprises reacting a controlled amount of hydrogen with the bio-farnesene in the presence of a catalyst under controlled reaction conditions. Any suitable hydrogenation catalyst may be used. For example, in some variations, a catalyst is selected from the group consisting of Pd, Pt, Ni, Ru, Ir, Cu, Fe, Raney-type porous catalysts such as Ni/Al, Co/Al and Cu/Al, alloys of platinum group catalysts with promoters or stabilizers such as Mo, Co, Mg and Zn, and hydroprocessing catalysts such as NiMoS and CoMoS. Exemplary catalysts are described in U.S. Pat. No. 6,403,844; U.S. Pat. No. 5,378,767; U.S. Pat. No. 5,151,172; and U.S. Pat. No. 3,702,348, each of which is incorporated herein by reference in its entirety. In certain embodiments, the controlled amount of hydrogen corresponds to a molar equivalent of desired degree of hydrogenation in the bio-farnesene. For example, to produce a 75% hydrogenated farnesene from the bio-farnesene, the controlled amount of hydrogen would be about 3 molar equivalents of hydrogen. Any suitable configuration for staged partial hydrogenation may be used to carry out the reaction with various catalyst conditions (e.g., structure of catalyst, type of catalyst, catalyst loading, reaction time, temperature and/or hydrogen pressure). For example, hydrogenations can be carried out in stages, a first stage, a second stage, and subsequence stages, if desired. The catalytic and hydrogenation conditions may be independently varied to produce partially hydrogenated farnesenes with a different degree of hydrogenation.

In certain embodiments, a composition comprising a high proportion of dihydrofarnesene may be selected for extraction of botanical compounds from plant materials. For example, the composition comprises at least about 85% of dihydrofarnesene, compared to the total amount of C15 hydrocarbons present in the composition. In certain embodiments, the compositions may comprise at least about 90% or at least about 95% dihydrofarnesene, compared to the total amount of C15 hydrocarbons present in the composition. Compositions comprising a relatively high proportion of dihydrofarnesene are particularly useful as solvents in extracting botanical compounds from plant materials.

The detailed description for producing farnesene, farnesene, and partially hydrogenated farnesene can be found in PCT Application Publication Nos. WO2012/141783 and WO2012/141784, which are incorporated herein by reference in their entirety. Additional description for producing different proportions of partially hydrogenated farnesenes can be found in U.S. Patent Application Publication No. 2015/0315520, which is incorporated herein by reference in its entirety. The bio-based farnesene is also commercially available and can be purchased from Amyris Inc. (Emeryville, Calif.). Farnesene and partially hydrogenated farnesene are also commercially available as, for example, Neossance® Hemisqualane and Myralene™ 10 Fluid from Amyris Inc. (Emeryville, Calif.).

### 6.3.2. C30 Hydrocarbon Compositions Derived from Bio-Based Farnesene

The bio-based farnesene derived from fermentation of renewable carbons can be used to generate C30 hydrocarbon compositions which are also suitable as carrier media for the present extraction methods. Examples of C30 hydrocarbons derivable from the bio-based farnesene include squalane, farnesene dimers, and farnesene dimers. In certain embodiments, the bio-based farnesene is chemically dimerized and then hydrogenated to produce squalane. The squalane composition provided herein can be differentiated from squalanes derived from shark oils or olive oils by the presence of isosqualane, which is co-produced with squalane from the catalytic reaction of bio-based farnesene substrates and subsequent hydrogenation. In certain embodiments, neosqualane and isosqualane are co-produced with squalane. In certain embodiments, the squalane composition derived from bio-based farnesene contains fewer impurities and/or lower quantities of impurities compared to squalanes obtained from shark oils or olive oils.

Any suitable catalysts may be used for the catalytic reaction to produce squalane and other C30 hydrocarbons from the bio-based farnesene. In certain embodiments, preformed or in situ-generated palladium catalysts can be used to catalyze the dimerization of bio-based farnesene to form a reaction product comprising isosqualene and structural isomers of isosqualene, and the reaction product can be hydrogenated to form a composition comprising squalane and isosqualane, and in some variations, also neosqualane. In contrast to a squalane composition derived from bio-based farnesene, squalane oils derived from olives or from shark liver do not comprise isosqualane.

In certain embodiments, palladium catalysts can be used to catalyze the dimerization of bio-based farnesene. In certain embodiments, the catalyst used herein is formed from a palladium precursor selected from [Pd(allyl)Cl]₂, Pd(cod)Cl₂, [Pd(allyl)Cl]₂, Pd(cod)Cl₂, Pd₂(dba)₃, Pd(dba)₂, Pd(dba), Pd(acac)₂, or an equimolar mixture of Pd(dba)₃ and Pd₂(dba)₃. In certain embodiments, the resulting catalyst comprises a phosphine ligand. In certain embodiments, the phosphine ligand is selected from triphenyl phosphine, triethyl phosphine and tritolyl phosphine.

In certain embodiments, dimerization of bio-based farnesene produces isosqualene, which can be subsequently hydrogenated to produce C30 hydrocarbon compositions. In certain embodiments, the hydrogenation reaction can be carried out in the presence of hydrogen with a catalyst such as Pd, Pd/C, Pt, Pt0₂, Ru(PPh₃)₃Cl₂, Rh(PPh₃)₃, Ru/C, Raney nickel, nickel, or combinations thereof. The hydrogenation reaction can be carried out as known to one of skill in the art, as reported in PCT Application Publication No. WO 2010/044208, which is incorporated herein by reference in its entirety.

Hydrogenated dimerization products resulting from these catalyst systems may be hydrocarbon compositions comprising squalane and isosqualane, wherein a ratio of (quantity squalane):(quantity isosqualane) is in a range from about 2:1 to about 26:1, e.g., 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1 or 26:1. In certain embodiments, the carrier medium consists of a C30 hydrocarbon composition (e.g., a squalane composition), obtained from bio-based farnesene, comprising at least about 85% by weight of squalane and equal to or less than about 15% by weight of isosqualane, based on the total weight of the C30 hydrocarbon composition. In certain embodiments, the carrier medium consists of a C30 hydrocarbon composition comprising at least about 90% by weight of squalane and equal to or less than about 10% by weight of isosqualane, based on the total weight of the C30 hydrocarbon composition. In certain embodiments, the carrier medium consists of a C30 hydrocarbon composition comprising from about 90% to about 98% by weight of squalane and from about 2% to about 10% by weight of isosqualane, based on the total weight of the C30 hydrocarbon composition.

Other suitable catalysts may be used if it is desired to produce squalane compositions comprising different proportions of squalane and isosqualane. For example, zirconium, titanium or hafnium catalysts can be used to catalyze the dimerization of bio-based farnesene to produce a reaction product, which, when hydrogenated, comprises isosqualane as the predominated product of squalane and isosqualane. The additional details about the catalysts, catalytic reactions and hydrogenation conditions are described in PCT Application Publication No. WO2011/146837, which is incorporated herein by reference in its entirety.

In another aspect, farnesene dimers and farnesene dimers may be used as a carrier medium. The farnesene dimers may be derived from bio-based farnesene using any suitable methods. For example, the bio-based farnesene may be heated to 220° C. and stirred to produce farnesene dimers. The farnesene dimers may be produced by reducing farnesene dimers in the presence of hydrogen with a catalyst such as Pd, Pd/C, Pt, PtO₂, Ru(PPh₃)₂Cl₂, Raney nickel, or combinations thereof. The detailed description for producing hydrocarbon compositions comprising farnesene dimers and farnesene dimers can be found in U.S. Pat. No. 7,592,295, U.S. Pat. No. 7,691,792, and U.S. Pat. No. 8,669,403, which are incorporated herein by reference in their entirety. The squalane derived from bio-based farnesene is also commercially available as Neossance® squalane from Amyris, Inc. (Emeryville, Calif.). The farnesene dimer and farnesene dimers are also commercially available from Amyris, Inc. (Emeryville, Calif.).

### 6.4 Personal Care Products and Kits

In another aspect, the present botanical infused products can be formulated as a variety of personal care products including cosmetics or perfume products. Because the botanical infused products provided herein utilize a carrier medium which is substantially free of impurities and odor, they provide significant benefits to personal care, cosmetics and perfume industry. The botanical infused products can be used to condition and enrich skin, hair, or nail, as a bath or massage oil, as cosmetics, as a fragrance, as cosmetics, as ointments, or as perfume. The botanical infused products may also be used for aromatherapy and for medicinal or homeopathic remedies to relieve pain, heal cuts, scrapes, or insect bites.

The personal care products can further contain additional ingredients other than botanical infused products. For example, the personal care consumer products can include a skin conditioning agent, such as humectants, exfoliants, emollients, and the like. The amount of skin-conditioning agent may range from about 1% to about 95%, about 5% to about 90%, about 10% to about 80%, or about 20% to about 60% of the total weight of the personal consumer products. Depending on the plant material used as a source to extract botanical compounds, personal care products may include anti-oxidant, anti-aging, skin brightening, or other beneficial properties. In certain embodiments, the personal care products may further comprise sunscreen agents. The botanical infused products provided herein are capable of uniformly dissolving physical sunscreen agents (e.g., zinc oxide or titanium oxide) and provide high spreadability on skin. The personal care products can be formulated in any suitable form, such as liquid, semi-liquid, suspension, cream, lotion, semi-solid, solid, impregnated substrate, or the like that can be topically applied to a consumer (e.g., skin, hair or nails).

In certain embodiments, a kit comprising a botanical infused product is provided. The kit may comprise infused products described herein and instructions for using the botanical infused products. For example, the kit embodiment may include instructions for applying infused products to skin, hair or nails. In another example, instructions may include directions for diluting infused products with a suitable diluent and appropriate dilution ratios. The kit may further comprise a wipe that is dry or pre-moistened with the present infused products.

### 6.5 Assays to Determine Quality and Quantity of Botanical Compounds in Infused Products

Any suitable assays known in the art may be used to determine the quality and quantity of botanical compounds present in the infused end products. There are at least five different parameters that can be used to evaluate odor from the infused end products. These parameters include hedonic tone, intensity, threshold value, persistency and characterization. Hedonic tone measures how well a given population likes an odor. Intensity measures the absolute intensity of an odor—how strong or weak it is. The odor threshold value is the concentration in air where an odor can first be detected. Odor persistency is a measure of how quickly a given odor dissipates in air. Characterization is a technique for describing an odor according to formal terminology with categories and subcategories. These parameters for evaluating an odor from a sample are further described in, for example, “Odor Basics”—Understanding Odor Testing,” McGinley et al., 22^{nd} Annual Hawaii Water Environment Association Conference, Honolulu, Hi. June 2000; “Odor Parameters” St. Croix Sensory, Inc. (2007).

In one embodiment, odor intensities of the presently provided infused end products may be characterized using ASTM E544 methods. ASTM E544, Standard Practice for Referencing Suprathreshold Odor Intensity, provides two methods for referencing the intensity of a sample of odorous air. These include: Procedure A—Dynamic Scale method and Procedure B—Static Scale Method. The Dynamic Scale Method uses a laboratory olfactometer device. Using the olfactometer device, a continuous flow of a standard odorant is provided for presentation to a panelist (assessor). The panelist compares the intensity of an odor sample to a specific concentration level of the standard odorant from the laboratory olfactometer device. The Static Scale method utilizes a set of bottles that include fixed dilutions of the standard odorant in a water solution.

Odor intensity quantification can be determined using an “Odor Intensity Referencing Scale” (OIRS). Odor intensity referencing compares the odor in the sample to the odor intensity of a series of concentrations of a reference odorant. Any suitable reference odorant can be selected. Additional details of the odor intensity quantification methods are described in “Odor Intensity Scales for Enforcement, Monitoring, and Testing,” C. M. McGInley, St. Croix Sensory Inc. and M. A. McGinley, McGinley Associates, P.A., Air and Waste Management Association, 2000 Annual Conference Session No: EE-6, Session Title: Odor Management and Regulation.

Alternatively or additionally, odor parameters of the botanical infused products can be assessed using electronic nose devices. The electronic nose devices (e.g., Cyranose® commercially available from Sensigent, Baldwin Park, Calif.) reproduce human senses using sensory arrays and pattern recognition system by measuring and analyzing volatile organic compounds in a gaseous sample. In embodiments of the present invention, the aroma signature from plant materials in their natural state can be first analyzed as a reference signature. The gas sample analysis from infused end products can then be compared to the known, reference signature to determine the quantity and quality of botanical compounds in the infused products. The botanical infused products obtained using different carrier media can be compared relative to one another to determine the relative quality and/or quantity of botanical compounds in the botanical infused products.

Other techniques for quantifying botanical compounds in infused products include gas chromatography (GC). If identities of botanical compounds in a plant material are known, then the quantity of botanical compounds in the botanical infused products produced by the present methods can be analyzed using gas chromatography-mass spectrometry (GC-MS) or by gas chromatography-flame ionization detection (GC-FID) methods. The relative area percentage (%) of the peaks in gas chromatograms representing botanical compounds in the botanical infused products can be compared against a calibration curve created using known quantities of reference compounds.

The assays described herein are merely exemplary, and other suitable assays may be used to analyze odor parameters of infused end products provided herein.

### 7. EXAMPLES

### 7.1 Example 1

This example provides comparison of infused products produced using different carrier media. Several different types of carrier media are used in this example: C30 hydrocarbon compositions obtained from bio-based farnesene (e.g., Neossance® Squalane), C15 hydrocarbon compositions obtained from bio-based farnesene (e.g., Neossance® Hemisqualane, Myralene™ fluids), squalane derived from olives, and other industry acceptable carrier oils. Neossance® Squalane and Neossance® Hemisqualane can be purchased from www.neossance.com. Squalane derived from 100% olives can be purchased from a number of different vendors (e.g., Squalane from Life-Flo of from Botanical Beauty).

For each carrier medium, an equal weight of fresh or dried flowers (e.g., lavender or rose petals) and squalane is mixed in a glass container. The mixture is incubated under solar radiation for at least three hours to several days with occasional shaking of the glass container.

The resulting infused products are shipped to St. Croix Sensory, Inc. for evaluation. A panel of experts at St. Croix Sensory, Inc. analyzes infused products and compares their intensities for lavender or rose aroma using ASTM E544 methods. The botanical infused products are also evaluated qualitatively by an odor panel in-house composed of at least 10 panelists.

### 7.2 Example 2

### 7.2.1. Experimental Method

Rose petals from Fragrant Cloud roses were cut into small pieces. In each 40 mL glass vial, 5 grams of cut petals were loaded. Thirty (30) mL of extraction oil (C30 hydrocarbon composition obtained from bio-based farnesene (e.g., Neossance® squalane), olive derived squalane, jojoba oil) was added in each separate vial with the rose petals. The vials were capped and shaken for about 1 minute, then incubated at 40° C. for 17 hours without shaking. At the end of 17 hours, the oils were filtered through 0.2 micron PTFE filters and stored in glass vials in a cool and dark environment until analysis.

### 7.2.2. Analysis and Methodology

An odor panel was conducted at a third party laboratory specialized in sensory and odor evaluations. Twenty one (21) assessors, who were trained and experienced at odor and taste evaluations, evaluated the samples based on floral intensity using a ranking test.

Five (5) mL of sample from each extraction oil was placed into amber glass wide mouth jars blinded with randomly generated three digit codes at ambient temperature and then covered with lids. Five minutes before each evaluation, the lids were removed, and the jars placed onto a tray to be presented to each assessor in a Latin square design balanced for position and carryover effects. Each assessor was asked to rank the samples by floral intensity. Assessors were instructed to evaluate them from left to right, and rank the samples from strongest to weakest floral intensity. Re-sniffing was allowed.

### 7.2.3. Results

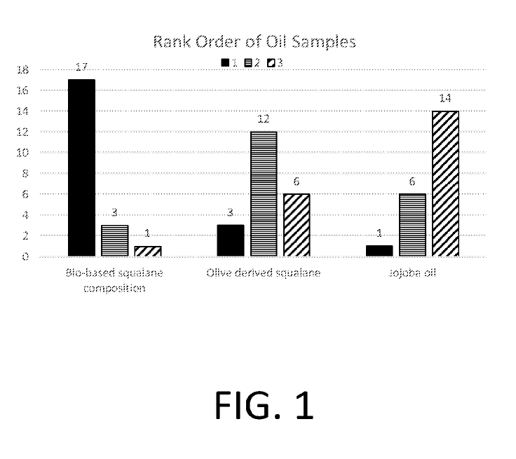

Table 1 and FIG. 1 provide the results of the ranking test of three extraction samples using three different carrier media: a squalane composition derived from bio-based farnesene (e.g., Neossance® squalane), a squalane oil from olives, and a jojoba oil. Twenty-one assessors participated in the ranking test. The results shown in Table 1 show the assessors' selection of sample rank by floral intensity from strongest (1) to weakest (3). Seventeen of the twenty-one assessors ranked a squalane composition derived from bio-based farnesene (e.g., Neossance® squalane) as the strongest of the three samples. Fourteen of the twenty-one assessors ranked jojoba as the weakest of the three samples.

TABLE 1

| | Squalane | | |
|---|---|---|---|
| | composition derived | Olive | |
| | from bio-based | derived | |
| Assessor | farnesene | squalane | Jojoba |
| 1 | 1 | 2 | 3 |
| 2 | 2 | 1 | 3 |
| 3 | 1 | 2 | 3 |
| 4 | 2 | 3 | 1 |
| 5 | 3 | 1 | 2 |
| 6 | 1 | 2 | 3 |
| 7 | 1 | 3 | 2 |
| 8 | 1 | 2 | 3 |
| 9 | 2 | 1 | 3 |
| 10 | 1 | 2 | 3 |
| 11 | 1 | 2 | 3 |
| 12 | 1 | 2 | 3 |
| 13 | 1 | 2 | 3 |
| 14 | 1 | 3 | 2 |
| 15 | 1 | 3 | 2 |
| 16 | 1 | 3 | 2 |
| 17 | 1 | 2 | 3 |
| 18 | 1 | 2 | 3 |
| 19 | 1 | 2 | 3 |
| 20 | 1 | 2 | 3 |
| 21 | 1 | 3 | 2 |
| Ranks | 26 | 45 | 55 |
| Sums | | | |

### 7.2.4. Rank Sum Test

The rank sum test is performed by totaling the ranks of each sample. The difference in rank sums needed in order to conclude there is a significant difference between the samples at the 95% confidence level for twenty-one assessors is 16. As outlined in Table 2, the difference between a squalane composition derived from bio-based farnesene (e.g., Neossance® squalane, also referred to as bio-based squalane) and olive derived squalane, and bio-based squalane and olive derived squalane, and bio-based squalane and jojoba oil meet this criteria. Such a result indicates that bio-based squalane is significantly stronger in floral intensity than both olive derived squalane and jojoba oil.

TABLE 2
Rank sum test of three carrier media: bio-based squalane as
a carrier medium to extract botanical compounds from rose petals,
olive derived squalane as a carrier medium to extract botanical
compounds from rose petals, and jojoba oil as a carrier medium
to extract botanical compounds from rose petals

| Rank Comparison | Calculation | Difference | Significance |
|---|---|---|---|
| Bio-based squalane vs | 48 − 26= | 19 | significant |
| Olive derived squalane | | | |
| Bio-based squalane vs | 55 − 26= | 29 | significant |
| Jojoba | | | |
| Olive derived squalane | 55 − 45= | 10 | not significant |
| vs Jojoba | | | |

### 7.2.5. Sign Test

The pairwise sign test is performed by comparing the ranking between each pair of samples and assigning a ‘+’ or ‘−’ to indicate which sample received a higher rank. The minimum number of judgments needed to establish significance at probability levels of 5% for twenty-one assessors is 15. As outlined in Table 3, all three sample pairs meet this criteria, meaning that bio-based squalane (e.g., Neossance® squalane) is significantly stronger in floral intensity than both olive derived squalane and jojoba oil, and olive derived squalane is significantly stronger in floral intensity than jojoba oil.

TABLE 3
Sign test of three carrier media: bio-based squalane as a carrier
medium to extract botanical compounds from rose petals, olive
derived squalane as a carrier medium to extract botanical compounds
from rose petals, and jojoba oil as a carrier medium to extract
botanical compounds from rose petals

| Rank Comparison | + | − | p-value | Significance |
|---|---|---|---|---|
| Bio-based squalane vs | 18 | 3 | 0.001 | significant |
| Olive derived squalane | | | | |
| Bio-based squalane vs | 19 | 2 | <0.001 | significant |
| Jojoba | | | | |
| Olive derived squalane | 15 | 6 | 0.039 | not significant |
| vs Jojoba | | | | |

### 7.2.6. Conclusions

According to the rank sum test, bio-based squalane (e.g., Neossance® squalane) is significantly stronger in floral intensity than both olive derived squalane and jojoba oil. According to the sign test, bio-based squalane is significantly stronger in floral intensity than both olive derived squalane and jojoba oil, and olive derived squalane is significantly stronger in floral intensity than jojoba oil.

Under the same extraction condition, bio-based squalane performs better in extracting botanical compounds, such as rose aroma, than both olive derived squalane and jojoba oil.

One or more features from any embodiments described herein may be combined with one or more features of any other embodiment described herein without departing from the scope of the invention.

All publications, patents and patent applications cited in this specification are herein incorporated by reference as if each individual publication or patent application were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

1. A method of extracting botanical compounds from a plant or plant material comprising:
(a) contacting a plant material comprising one or more botanical compounds with a carrier medium to produce a mixture, wherein the carrier medium comprises a bio-based farnesene, a hydrocarbon composition derived from the bio-based farnesene, or a combination thereof; and
(b) incubating the mixture to extract the botanical compounds from the plant material into the carrier medium to produce a botanical infused product comprising the botanical compounds.

2. The method of claim 1, wherein the carrier medium comprises a C15 hydrocarbon, C30 hydrocarbon, or a combination thereof.

3. The method of claim 2, wherein the carrier medium comprises farnesene, partially hydrogenated farnesene, squalane, farnesene dimer, farnesene dimer, or any combination thereof.

4. The method of claim 3, wherein the carrier medium comprises squalane.

5. The method of claim 4, wherein the carrier medium further comprises isosqualane.

6. The method of claim 5, wherein the proportion of squalane to isosqualane in the carrier medium is between about 2:1 to about 25:1.

7. The method of claim 1, wherein the carrier medium comprises a partially hydrogenated farnesene.

8.-9. (canceled)

10. The method of claim 1, wherein the plant material is selected from a whole plant, stem, flower, root, seeds, fruit, leaf, bark, carpels, stamen, petals or any combination thereof, or wherein the plant material is selected from rose petals, jasmine flowers, orange blossom, grapefruit blossom, lime blossoms, nectarine blossom, calendula, calendula flowers, peony, lily, bluebell, lavender, gardenia, marigold, daffodil, verbena, linden, chamomile, geranium, basil, sage, ginger, ginseng, cacao, nutmeg, anise, sage, and vanilla beans.

11. (canceled)

12. The method of claim 1, wherein the one or more botanical compounds comprise a volatile odorous compound, or wherein the one or more botanical compounds are useful for medicinal or homeopathic remedies.

13. (canceled)

14. The method of claim 1, wherein the plant material is at least partially dried prior to contact with the carrier medium or is pulverized.

15.-16. (canceled)

17. The method of claim 1, wherein the mixture is incubated at an elevated temperature greater than 30° C., or wherein the mixture is incubated under solar radiation.

18. (canceled)

19. The method of of claim 1, wherein the mixture is incubated at a pressure greater than atmospheric pressure, or wherein the mixture is incubated at a temperature between about 4° C. to about 15° C.

20.-22. (canceled)

23. The method of claim 1, wherein the plant material is rose petals.

24. The method of claim 1, wherein intensity of fragrance of the botanical infused product is significantly greater than that prepared using an olive derived squalane as a carrier medium.

25. (canceled)

26. The method of claim 1, wherein the carrier medium comprises a squalane composition comprising from about 90% to about 98% by weight of squalane and from about 2% to about 8% by weight of isosqualane, based on the total weight of the squalane composition.

27. A botanical infused product produced by the method of claim 4.

28. A botanical infused product comprising:
(a) one or more botanical compounds extracted from a plant material; and
(b) a carrier medium comprising a bio-based farnesene or a C15 or C30 hydrocarbon composition produced from the bio-based farnesene.

29. (canceled)

30. The botanical infused product of claim 28, wherein the carrier medium comprises squalane.

31. The botanical infused product of claim 30, wherein the carrier medium further comprises isosqualane.

32.-38. (canceled)

39. A personal care product comprising the botanical infused product of claim 28, which is formulated as a skin, hair, sun care, cosmetic and cleansing product.
